(19) United States
(12) Patent Application Publication
(10) Pub. No.: US 2023/0255642 A1
(43) Pub. Date: Aug. 17, 2023

(54) WEARABLE COMPRESSION DEVICE

(71) Applicant: INNOVATION LAB. LLC, La Palma, CA (US)

(72) Inventors: Eric Nathan Bowman, Franklin, TN (US); Orr Limpisvasti, Manhattan Beach, CA (US); Myong Kim, Tustin, CA (US); Hieu Van Le, Fountain Valley, CA (US); Joseph Paul Randolph, Seal Beach, CA (US)

(21) Appl. No.: 18/006,377

(22) PCT Filed: Jul. 6, 2021
(86) PCT No.: PCT/US2021/040565

§ 371 Date: Jan. 20, 2023

Related U.S. Application Data
(60) Provisional application No. 63/054,586, filed on Jul. 21, 2020.

(51) Int. Cl. A61B 17/135; A61B 17/132

(52) U.S. Cl. CPC A61B 17/1355; A61B 17/1325

(57) ABSTRACT

Disclosed are devices, systems, and methods of a wearable compression control system for actively controlling an applied pressure against an extremity to restrict blood flow along the extremity of a user performing an exercise or undergoing a therapy. In some embodiments, the system can include an inflatable band that extends circumferentially around the extremity. The inflatable band can include a bladder having an expandable inner volume for receiving fluid to cause the inflatable band to increase in thickness and increase an applied pressure against the extremity. The system can further include a controller that releasably couples to the inflatable band and includes a processor having a programmed pressure range. The processor can be configured to actively maintain the applied pressure within the programmed pressure range throughout the user performing the exercise or the user undergoing the therapy.

### CROSS-REFERENCE TO RELATED APPLICATION

This application claims priority under 35 U.S.C. § 119(a) to U.S. Provisional application Ser. No. 63/054,586, filed on Jul. 21, 2020 and entitled “WEARABLE COMPRESSION DEVICE,” the disclosure of which is incorporated by reference herein in its entirety.

### TECHNICAL FIELD

The subject matter described herein relates to various embodiments of a wearable, controllable compression device.

### BACKGROUND

Muscular strength and hypertrophy can be developed through resistance training. For example, muscular strength improvements may be achieved through resistance training at an intensity of 60-80% of an individual's one-repetition maximum strength (1-RM). However, such resistance training intensity can be impossible or harmful for some people. For example, a person having a post-acute injury, a post-operative condition, or certain chronic diseases can be injured attempting to gain strength through intense resistance training.

Compression therapy, or blood flow restriction therapy, can aid in improving muscular strength in people who cannot tolerate higher intensity resistance training. For example, blood flow restriction therapy may include performing low-intensity exercise (e.g., involving an arm) while restricting blood flow (e.g., to the arm), such as by using a tourniquet to restrict blood flow (e.g., to the arm). For example, a tourniquet can be used to restrict blood flow to an arm to allow the arm to perform lower-intensity exercise while developing muscular strength that is approximately the same as if the arm was performing a higher-intensity exercise. At least some drawbacks associated with use of a tourniquet for blood flow restriction at least for use with exercise or other compression-based therapies (e.g., ischemic preconditioning) can include the cost of the tourniquet, limited range of mobility of the user at least partially due to limited portability, and difficulty applying and maintaining a desired/beneficial amount of pressure. Thus, reliable and efficient devices, methods, and systems for restricting blood flow during exercise or compression therapy are needed.

### SUMMARY

Aspects of the current subject matter include various embodiments of a wearable compression system that can actively control compression along an extremity of a user. In one aspect, a wearable compression control system for controlling an applied pressure against an extremity to restrict blood flow along the extremity of a user performing an exercise or undergoing a therapy is described herein. For example, the wearable compression control system can include an inflatable band including a coupling feature for securing the inflatable band in a position along the extremity such that the inflatable band extends circumferentially around the extremity. The inflatable band can include a bladder extending along a length of the inflatable band. The bladder can include an expandable inner volume for receiving fluid to cause the inflatable band to increase in thickness and increase the applied pressure against the extremity. The wearable compression control system can further include a controller that releasably couples to the inflatable band. The controller can include a processor including a programmed pressure range. The processor can be configured to actively maintain the applied pressure within the programmed pressure range throughout the user performing the exercise or the user undergoing the therapy. The controller can further include a pressure sensor positioned along an airflow pathway that is in fluid communication with the bladder, the pressure sensor can sense a pressure within the bladder that indicates an amount of the applied pressure against the extremity for at least restricting blood flow. The processor can be configured to receive sensed pressure data from the pressure sensor during performance of the exercise or therapy. The controller can further include a pump in fluid communication with the airflow pathway. The pump can be configured to continuously receive instructions from the processor to provide fluid to the bladder for increasing the pressure within the bladder to reach the programmed pressure range throughout the user performing the exercise or the user undergoing the therapy. The controller can further include a relief valve configured to receive instructions from the processor to release fluid from the bladder to thereby reduce the pressure within the bladder when the sensed pressure is above the programmed pressure range.

In some variations one or more of the following features can optionally be included in any feasible combination. The controller can include a controller coupling interface and the inflatable band can include a band coupling interface. The controller coupling interface can include a first coupling feature that releasably engages with a second coupling feature of the band coupling interface for releasably coupling the controller to the inflatable band. The coupling of the band coupling interface to the controller coupling interface can form a part of the airflow pathway extending between the pump and the bladder. The band coupling interface can include a magnet and the controller can include a hall effect sensor for detecting when the band coupling interface is adjacent the controller coupling interface. The programmed pressure range can be approximately 40% to approximately 80% of a limb occlusion pressure associated with the user.

The controller can further include a user interface. The user interface can include a touchscreen. The controller can further include a rechargeable battery. The controller can further include an accelerometer that measures acceleration of the wearable compression system during the user performing the exercise or the user undergoing the therapy. The processor can be configured to analyze the measured acceleration for determining whether the exercise was performed in accordance with one or more parameters. The controller can further include a light strip extending along at least one side of a housing of the controller. The light strip can provide an indication of one or more conditions of the wearable compression system and/or the user.

In another interrelated aspect of the current subject matter, a method includes sensing, by a pressure sensor positioned along a fluid pathway of the wearable compression system, a first sensed pressure indicating a first pressure within an inflatable band of the wearable compression system. The inflatable band can extend circumferentially around an extremity of a user. The method can further include receiving, at a processor of a controller of the wearable compression system, the first sensed pressure and comparing, at the processor, the first sensed pressure against a programmed pressure range. The programmed pressure range can include a lowest pressure for achieving blood flow restriction by the inflatable band along a part of the extremity.

The method can further include activating, when the processor determines the first sensed pressure is below the programmed pressure range, a pump of the wearable compression system to deliver fluid to the inflatable band for increasing the pressure within the inflatable band. The method can further include monitoring, by the processor and during activation of the pump, sensed pressure data from the pressure sensor and deactivating, by the processor, the pump when the processor receives a second sensed pressure that is greater than the programmed pressure range. The method can further include monitoring, by the processor and during performance of an exercise using the extremity, sensed pressure data from the pressure sensor and activating, when the processor determines a third sensed pressure is above the programmed pressure range, a relief valve to release fluid from the inflatable band. The method can further include actively controlling, by the processor during performance of the exercise, the pressure within the inflatable band to maintain the pressure within the programmed pressure range.

In some variations one or more of the following features can optionally be included in any feasible combination. For example, the controller can be configured to be releasably coupled to the inflatable band. Actively controlling the pressure within the inflatable band during performance of the exercise can allow the inflatable band to maintain blood flow occlusion during performance of the exercise. The programmed pressure range can be approximately 40% to approximately 80% of a limb occlusion pressure associated with the user. The method can further include displaying, at a user interface of the wearable compression system, one or more characteristics associated with an amount of pressure applied to the extremity by the inflatable band. The user interface can include a touchscreen. The controller can include a controller coupling interface and the inflatable band can include a band coupling interface. The controller coupling interface can include a first coupling feature that releasably engages with a second coupling feature of the band coupling interface for releasably coupling the controller to the inflatable band. The coupling of the band coupling interface to the controller coupling interface can form a part of the airflow pathway extending between the pump and the bladder. The band coupling interface can include a magnet and the controller can include a hall effect sensor for detecting when the band coupling interface is adjacent the controller coupling interface.

The controller can further include an accelerometer that measures acceleration of the wearable compression system during performance of the exercise, and the processor can be configured to analyze the measured acceleration for determining whether the exercise was performed in accordance with one or more parameters. The controller can further include a light strip extending along at least one side of a housing of the controller, and the light strip can provide an indication of one or more conditions of the wearable compression system and/or the user.

The method can further include calibrating a limb occlusion pressure of the user to determine the programmed pressure range. The method can further include sensing a movement of the user and tracking a number of movements performed by the user. The method can further include maintaining the applied pressure within the programmed pressure range during performance of the movements by the user.

The details of one or more variations of the subject matter described herein are set forth in the accompanying drawings and the description below. Other features and advantages of the subject matter described herein will be apparent from the description and drawings, and from the claims. The claims that follow this disclosure are intended to define the scope of the protected subject matter.

### BRIEF DESCRIPTION OF THE DRAWINGS

The accompanying drawings, which are incorporated in and constitute a part of this specification, show certain aspects of the subject matter disclosed herein and, together with the description, help explain some of the principles associated with the disclosed implementations. In the drawings,

FIG. 1A shows a perspective side exploded view of a wearable compression system wearable compression system including a controller and an inflatable band consistent with implementations of the current subject matter;

FIG. 1B shows a perspective side view of the wearable compression system of FIG. 1A;

FIG. 1C shows a side cross-section view of a coupling interface of the wearable compression system of FIG. 1A;

FIG. 2 shows a top view of an embodiment of the inflatable band including a bladder;

FIG. 3A shows a front view of an embodiment of the controller including a user interface;

FIG. 3B shows a side view of the controller of FIG. 3A;

FIG. 3C shows a front view of the controller of FIG. 3A with a part of a housing of the controller hidden;

FIG. 3D shows a side view of the controller of FIG. 3A with a part of a housing of the controller hidden;

FIG. 4 shows a side perspective view of an embodiment of a band coupling interface including at least one magnet;

FIG. 5A shows a side perspective view of an embodiment of a coupling interface including an embodiment of the band coupling interface coupled to a controller coupling interface;

FIGS. 5B-5D shows side perspective views of the coupling interface of FIG. 5A showing the band coupling interface uncoupling from the controller coupling interface;

FIG. 6A shows another embodiment of the coupling interface;

FIG. 6B shows a perspective cross-section view of the coupling interface of FIG. 6A;

FIG. 7A shows a side perspective exploded view of another embodiment of the wearable compression system including a controller and an inflatable band consistent with implementations of the current subject matter;

FIG. 7B shows a side perspective view of the wearable compression system of FIG. 7A;

FIG. 7C shows the wearable compression system of FIG. 7A coupled to an arm of a user;

FIG. 8A shows an exemplary mobile user interface for communicating with the controller of an embodiment of the wearable compression system;

FIG. 8B shows another exemplary mobile user interface display;

FIG. 9A shows an exemplary touchscreen user interface of the controller of an embodiment of the wearable compression system;

FIG. 9B shows another exemplary touchscreen user interface displaying various information that can be adjusted by a user;

FIG. 10 shows a flowchart illustrating a process for controlling a pressure inside of an inflatable band, in accordance with some example embodiments;

FIG. 11 shows a block diagram illustrating a computing system, in accordance with some example embodiments;

FIG. 12A shows an exemplary mobile user interface of a mobile device for communicating with the controller of an embodiment of the wearable compression system, the mobile user interface showing an embodiment of a user login display;

FIG. 12B shows another display of the mobile user interface of FIG. 12A showing selectable icons and information for pairing the mobile device with an embodiment of the wearable compression system;

FIG. 12C shows another display of the mobile user interface of FIG. 12A showing editable protocols that can be selected for controlling various parameters or characteristics of applied compression by the wearable compression system;

FIG. 12D shows another display of the mobile user interface of FIG. 12A showing an editable number of movement repetitions that are to be performed by a user of the wearable compression system during each set;

FIG. 12E shows another display of the mobile user interface of FIG. 12A showing calibrating occlusion pressure of a user of the wearable compression system;

FIG. 12F shows another display of the mobile user interface of FIG. 12A showing a summary of programmed parameters or characteristics associated with a programmed protocol of the wearable compression system;

FIG. 12G shows another display of the mobile user interface of FIG. 12A showing a summary of programmed parameters or characteristics associated with a programmed protocol of the wearable compression system and a status of programmed protocol; and

FIG. 12H shows another display of the mobile user interface of FIG. 12A showing with a system support display for an embodiment of the wearable compression system.

When practical, similar reference numbers denote similar structures, features, or elements.

### DETAILED DESCRIPTION

The present disclosure describes various embodiments of a wearable compression system that is effective for actively controlling an amount of applied pressure to an extremity of a user in order to appropriately restrict blood flow along at least a part of the extremity. Such active control of the amount of pressure to restrict blood flow can be performed during various levels of resistance training intensity (including low intensity) to increase muscle mass along the extremity. Additionally, the wearable compression system described herein can be used to treat various muscular, intravenous, and nervous system deficiencies. For example, the wearable compression system can provide non-invasive compression methods applied to the upper and/or lower extremities of the human body. For example, the wearable compression system may be worn on a user's arm or leg for the purpose of reducing blood flow to muscles distal to the wearable compression system to achieve one or more of the benefits disclosed herein, such as increased muscle mass achieved using less resistance against such muscles. Other potential medical or therapeutic applications or benefits of the system include global protection of various tissues or organs against ischemia by reperfusion of blood flow.

For example, reduced venous return blood flow in muscles distal to the wearable compression system can have the same or similar effect as high-intensity workout routines for inducing muscle hypertrophy and increased strength. For example, blood flow resistance therapy training at intensities as low as 20% of an individual's one-repetition maximum strength results in significant improvements in muscular strength and hypertrophy. The wearable compression system may be used for therapeutic and strength training purposes. For example, in orthopedics, non-surgical and post-surgical conditions may benefit from blood flow restriction therapy using the wearable compression system, such as to shorten recovery time and reduce muscle loss (atrophy).

The blood flow restriction methods and related wearable compression system disclosed herein can actively regulate pressure applied to an extremity, such as during exercise and/or therapy, thereby enhancing the effects of blood flow restriction and maximizing patient comfort and results. For example, regulating pressure applied by the wearable compression system can prevent discomfort and at least reduce excessive pressure exerted on the user. Furthermore, regulating pressure can ensure that the pressure applied to the extremity of the user does not drop above or below a desired pressure and/or pressure range. For example, the desired pressure and/or pressure range can be predefined and can define an amount of pressure required to achieve safe and effective blood flow restriction. In some embodiments, the applied pressure can be maintained in real-time to prevent loss of pressure between intervals. In some embodiments, the wearable compression system may regulate the applied pressure according to a limb occlusion pressure (e.g., a minimum amount of compressive forces or pressure that is needed to stop arterial blood flow past the wearable compression system). Limb occlusion pressure is user dependent and can vary among users of the wearable compression system. In some embodiments, the wearable compression system can include a programmed protocol for determining a limb occlusion pressure of a user, such as sense an amount of pressure to stop blood flow past the wearable compression system. Once the limb occlusion pressure is determined of a user, a percentage of the limb compression force can be programmed. For example, the wearable compression system can apply a pressure on an extremity that is between approximately 40% to approximately 80% of the limb occlusion pressure associated with the user.

The wearable compression systems and methods disclosed herein can be more compact and mobile than other blood flow restriction devices. For example, in some embodiments the wearable compression system can include an inflatable band and a controller that is releasably coupled to the inflatable band. The controller can include a user interface and/or display and can provide fluid (e.g., air) to the inflatable band for inflating the inflatable band to thereby apply a pressure to an extremity. For example the compactness and mobility of the wearable compression systems described herein can enable use in gym, home, clinical and/or physical therapy environments. Additionally, the wearable compression systems may be fully operational through the controller and/or may be wirelessly controlled (e.g., Bluetooth, Wi-Fi, etc.).

In some embodiments, the controller includes a pump configured to adjust an amount of fluid inside the inflatable band to adjust a compression force exerted by the inflatable band on a user. The controller can also include a pressure sensor configured to detect a pressure of an interior volume of the inflatable band. The controller can further include a processor configured to receive sensed pressure associated with the interior volume of the inflatable band, determine whether the pressure of the interior volume satisfies a threshold, and generate an instruction to the pump to adjust the amount of fluid inside the interior volume of the inflatable band to achieve a pressure that satisfies the threshold. In some embodiments, the processor can be configured to send instructions to a relief valve for releasing fluid from the interior volume of the inflatable band, such as to reduce the amount of pressure in the interior volume and/or reduce an amount of applied pressure on an associated extremity of a user. Various embodiments and methods associated with the wearable compression system is described herein.

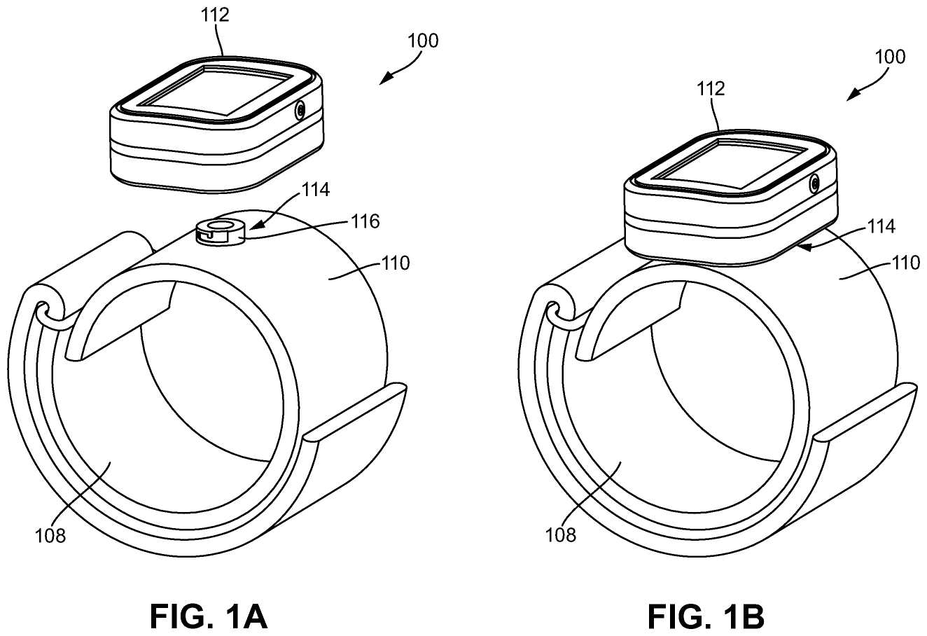

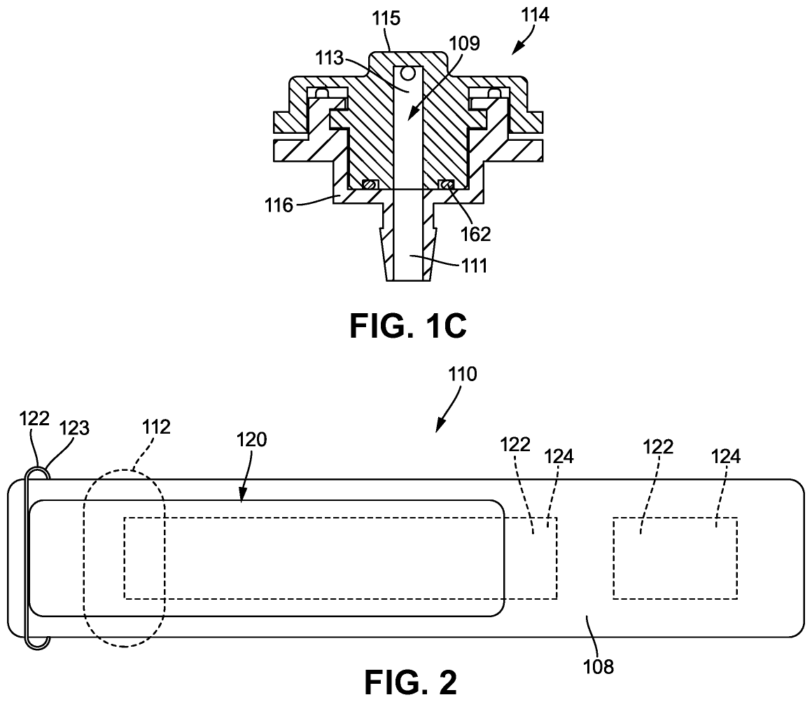

FIGS. 1A-1C illustrate an embodiment of a wearable compression system 100 for actively regulating and maintaining a pressure applied by the wearable compression system 100 to an extremity (e.g., arm, leg) of a user. Such active regulation of the applied pressure can achieve a desired amount of blood flow restriction to a portion of the extremity distal to the applied pressure, such as during performance of an exercise to achieve improved muscle strength under lower intensities experienced by the user (e.g., lower resistance exercise). As shown in FIGS. 1A-1B, the wearable compression system 100 may include an inflatable band 110 and a controller 112. The inflatable band 110 and the controller 112 may be configured to releasably couple to one another, such as via a coupling interface 114, as shown in FIG. 1C. As shown in FIGS. 1A and 1C, the controller 112 can include a controller coupling interface 115, which can be positioned along a side of the controller 112 facing the inflatable band 110 when the controller 112 is coupled to the inflatable band 110. Additionally, the inflatable band 110 can include a band coupling interface 116, as shown in FIGS. 1A and 1C. For example, the controller coupling interface 115 and the band coupling interface 116 can include one or more mating features for allowing the controller coupling interface 115 to releasably couple to the band coupling interface 116 thereby releasably coupling the controller 112 to the inflatable band 110. Any number of a variety of mating features can be included in the coupling interface 114 for allowing the controller 112 to releasably couple to the inflatable band 110 via the band coupling interface 116, some of which are described in greater detail herein.

In some embodiments, the coupling interface 114 can provide fluid communication between the controller 112 and the inflatable band 110, such as to allow the controller 112 to provide fluid flow to the inflatable band 110 to thereby inflate the inflatable band 110 (e.g., form an inflated configuration). When the wearable compression system 100 is coupled to an extremity, such as when the inflatable band 110 is secured around a circumference of an extremity, inflation of the inflatable band 110 can cause at least a part of the inflatable band 110 (e.g., an inner surface 108 of the inflatable band that is in contact with the extremity) to apply a pressure to the extremity. For example, this can be due to the inflatable band 110 increasing in thickness and decreasing an inner diameter of the inflatable band 110 extending circumferentially around the extremity. As discussed above, pressure applied to the extremity can cause blood flow restriction along the extremity, such as by reducing and/or blocking one or more blood flow pathways extending along the extremity.

In some embodiments, the inflatable band 110 may be included in a garment that is wearable on the human body. For example, as shown in FIGS. 1A-1B, the inflatable band 110 may include an extended piece of fabric that may be long enough to wrap around one or more human extremities, such as a part of an arm and/or leg. The inflatable band 110 may be made out of one or more of a variety of materials, such as a nylon, a poly cotton material, waterproof, and/or water-resistant materials. For example, the inflatable band 110 may include multiple layers or pieces of fabric sewn together, such that an interior volume is defined by at least two pieces of fabric.

FIG. 2 illustrates an embodiment of an inflatable band 110 including a contained inner volume or bladder 120 configured to contain fluid delivered from an embodiment of the controller 112 thereby allowing the inflatable band 110 to expand to apply a pressure to restrict blood flow along an extremity. For example, the bladder 120 can include a substantially sealed flexible and/or expandable container that is in fluid communication with the band coupling interface 116 for allowing fluid to be delivered and removed from the bladder 120. As shown in FIG. 2, the bladder 120 can extend along a length of the inflatable band 110. For example, the bladder 120 can extend between a first end of the inflatable band 110 and approximately half to approximately three-quarters the length of the inflatable band 110. The bladder 120 can have a rectangular shape or any of a variety of shapes. The bladder 120 can expand to increase a thickness of the inflatable band as a result of an increase in fluid volume deposited in the bladder 120.

The bladder 120 can be sewn and/or inserted into material forming the inflatable band 110. For example, the bladder 120 may be a flat design when empty (e.g., the inflatable band is in the deflated configuration) and expand in one or more directions when filled with a fluid (e.g., air). The bladder 120 can include a polyurethane or similar polymeric coating that can assist with providing an air seal and prevent leaks.

For example, when the wearable compression system 100 is secured to an extremity of a user, as the bladder 120 is filled with air, the inner volume of the bladder 120 and overall size of the bladder 120 can increase thereby enlarging a thickness of the inflatable band 110. For example, the thickness of the inflatable band 110 can increase such that an inner diameter of the inflatable band 110 decreases thereby increasing a pressure applied by the inner surface 108 of the wearable compression system 100 against the extremity along which the wearable compression system 100 is coupled to. The bladder 120 can be in fluid communication with the band coupling interface 116 such that when the controller 112 is coupled to the inflatable band 110, the bladder 120 can be in fluid communication with the pump of the controller 112. As such, output from the pump (e.g., airflow) can be delivered along an airflow pathway 109 that extends between the pump and the bladder 120, as shown in FIG. 1C.

For example, as shown in FIG. 1C, the airflow pathway 109 can include a bladder airflow pathway 111 extending through the band coupling interface 116 and a controller airflow pathway 113 that extends through the controller coupling interface 115. When the coupling interface 114 is formed (e.g., the controller coupling interface 115 is releasably coupled to the band coupling interface 116), the airflow pathway 109 extending between the pump of the controller 112 and the bladder 120 can be formed. Any number of a variety of air-sealing features can be included in the coupling interface 114 to prevent fluid leaks along the airflow pathway 109. For example, one or more o-rings 162 can be positioned between one or more sides of the band coupling interface 116 and the controller coupling interface 115 to prevent fluid leaks therebetween.

The inflatable band 110 may include one or more coupling features 122 for adjusting and/or securing a length of the inflatable band 110 extending around an extremity, such as securing the wearable compression system 100 securely in a position along the extremity for restricting blood flow. As shown in FIG. 2, the inflatable band 110 can include more than one coupling features 122, such as a ring 123 (e.g., D-ring) and a securing feature 124 (e.g., Velcro, hook and loop, etc.) that secures a formation of the inflatable band 110 (e.g., around an extremity). For example, the first end of the inflatable band 110 can include the ring 123 at or adjacent the first end. In addition, one or more securing features 124, such as Velcro, can be positioned at and/or adjacent an opposing second end of the inflatable band 110. Securing features 124, such as Velcro and/or clasps, can be positioned along a length of the inflatable band 110, such as shown in FIG. 2. For example, the second end can be advanced through the ring 123 and folded over a part of the ring 123 to allow the securing feature 124 adjacent the second end to secure (e.g., using Velcro) to the inflatable band 110 between the first and second ends.

The inflatable band 110 can be secured along an extremity such that the inflatable band 110 stays in position along the extremity before and after inflation of the inflatable band 110. The coupling features 122 and securing features 124 can include one or more of a variety of features that releasably secure the inflatable band 110 to an extremity, including during and after inflation of the inflatable band 110. For example, the coupling features 122 and/or securing features 124 can include one or more of a glue, Velcro, a latch and hook assembly, a tape, a magnet, adhesive, tab and insert, staples, stitches, and/or the like. For example, the ring 123 can allow for efficient self-application of the wearable compression system 100 to an extremity, such as to an arm.

The inflatable band 110 can be shaped to conform to a human extremity, such as an arm and/or leg. In some embodiments, the deflated inflatable band 110 may be a flat, planar cloth that may be folded or wrapped around a human appendage. As shown in FIG. 2, the inflatable band 110 can have a rectangular shape, however, other shapes are within the scope of this disclosure. The inflatable band 110 may be made of one or more of a fabric, plastic, leather, faux-leather, and/or the like. The inflatable band 110 may be tapered to improve the comfort and uniformity of the pressure distribution across the section of the extremity. The length and width of the inflatable band 110 may be adjusted according to the extremity. For example, an approximately 3 inch wide inflatable band 110 may be used for an arm, and another embodiment having an approximately 4 inch wide inflatable band 110 may be used for a leg.

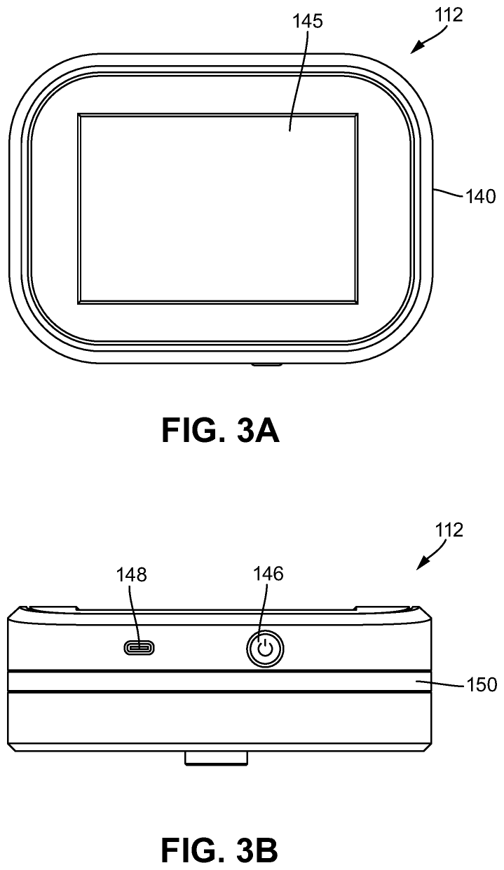

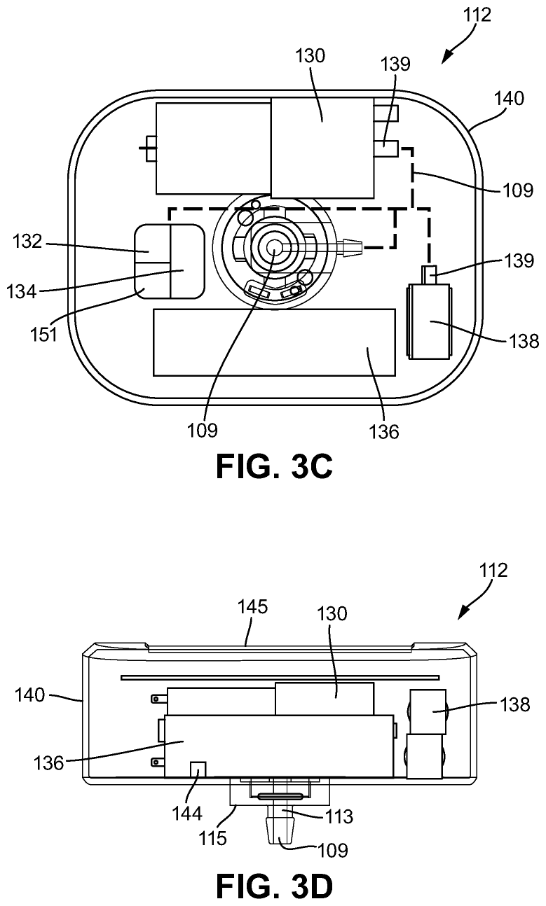

FIGS. 3A-3D illustrate an embodiment of the controller 112 of the wearable compression system 100 that is configured to actively regulate and maintain an amount of pressure applied to an extremity, which can be determined by sensing a pressure within the inflatable band 110. For example, the controller 112 may regulate the pressure inside of the inflatable band 110 by adjusting the amount of fluid (e.g., air) inside the bladder 120 of the inflatable band 110. By adjusting the amount of fluid inside the bladder 120 of the inflatable band 110, the pressure applied by the inflatable band 110 (e.g., via the inner surface 108 against the user) can be adjusted. For example, an increase in fluid in the bladder 120 can result in increased applied pressure, and a decrease in fluid in the bladder 120 can result in a decrease in applied pressure. The amount of applied pressure provided by the inflatable band 110 to a user may be proportional to the pressure inside the bladder 120 of the inflatable band 110.

As shown in FIGS. 3C and 3D, the controller 112 can include one or more of a pump 130, a pressure sensor 132, a relief valve 138, and a processor 134 for controlling the pressure inside of the inflatable band 110. For example, the pressure sensor 132 can be in contact and/or communication (e.g., fluid communication) with the bladder 120 to thereby enable the pressure sensor 132 to sense a pressure within the bladder 120. The pressure sensor 132 can be in communication with the processor 134 which can analyze the sensed pressure data. For example, the processor 134 may compare the sensed data against predefined pressure values and/or ranges to determine whether the pressure in the bladder 120 should be increased, decreased or not adjusted in order to achieve a desired amount of applied pressure on the user for achieving a desired amount of blood flow restriction during performance of an exercise by the user.

For example, the processor 134 can be programmed (e.g., by a user, a clinician, and/or programmed prior to sale) with predefined pressure values and/or ranges that define a threshold or desired pressure value to achieve a desired amount of applied pressure, such as to achieve blood flow restriction along the respective extremity to increase strength of the extremity. For example, the processor 134 of the controller 112 may be in communication with and generate an instruction for the pump 130 to adjust the amount of fluid inside the bladder 120 in response to the sensed pressure in order to satisfy the programmed pressure values and/or ranges. For example, if the processor 134 determines that sensed pressure from the pressure sensor 132 is below a programmed pressure value threshold defining an amount of pressure required to restrict blood flow, the processor 134 can send an instruction to the pump 130 to deliver more fluid to the bladder 120 to increase the pressure within the bladder 120. The pump 130 can continue to deliver fluid to the bladder 120 until the sensed pressure satisfies the programmed pressure values and/or ranges.

As shown in FIGS. 3A-3D, the controller 112 may include a housing 140 that provides an outer protective shell to contain at least the pump 130, the pressure sensor 132, and the processor 134. The housing 140 may include a front side with a user interface 145 and a backside including the controller coupling interface 115, however, the controller coupling interface 115 and the user interface 145 may be positioned on any one or more sides of the housing 140 without departing from the scope of this disclosure.

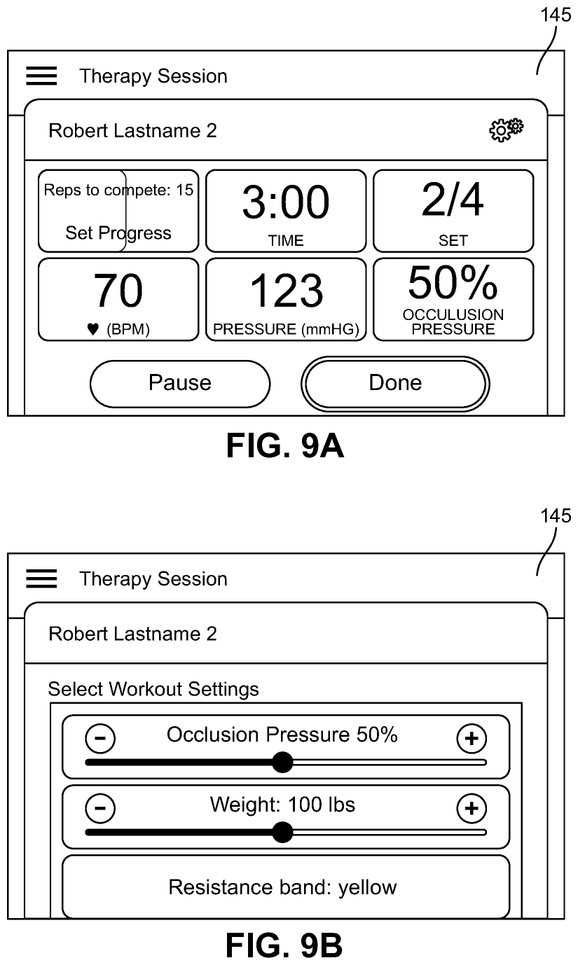

For example, the user interface 145 may include a screen to display readings relating to the inflatable band 110. For example, the user interface 145 may display the sensed pressure within the bladder 120. The user interface 145 may include a touchscreen that enables the user to select settings related to the inflatable band 110 (as shown, for example, in FIGS. 9A and 9B). For example, the user interface 145 may present a menu from which the user may select a percentage of occlusion pressure, enabling the user to set pressure thresholds of the interior volume. In some embodiments, the user interface 145 can rotate a display along the user interface 145, such as to ensure the display is positioned upright in accordance with user viewing (e.g., standing upright). For example, the display can automatically rotate depending on an orientation of the user's arm relative to the ground. For example the controller 112 can include an inertial measurement unit (IMU) that can assist with appropriately rotating the display of the user interface 145.

Alternatively, and/or additionally, the controller 112 can include one or more tactile buttons that enable the user to select settings related to the inflatable band. As shown in FIG. 3B, the controller 112 can include a tactile power button 146 along a side of the controller 112. The same or different side of the controller 112 can also include a charging port 148 for connecting the controller 112 to a power source, such as to recharge a rechargeable battery 136 of the controller 112, as shown in FIGS. 3B and 3C. In some embodiments, the charging port 148 can be used for downloading programs and/or information, such as exercise therapy data that can be downloaded from the controller 112. One or more exercise/therapy routines can be uploaded to the controller 112 via the charging port 148.

The controller 112 can include any number of sensors for sensing, measuring, and/or analyzing a variety of data. For example, the controller 112 can include one or more sensors for collecting sensed data in order to anticipate or determine the pressure inside of the inflatable band 110. Examples of such sensors can include an accelerometer 151 and/or a blood pressure sensor, which can communicate sensed data to the processor 134. Other data can be input by a user and/or sensed, such as a duration of an exercise, a surface area covered by the inflatable band 110, and/or a diameter of the limb around which the inflatable band 110 is wrapped. The controller 112 can monitor the pressure inside of the inflatable band 110 continuously or at a time interval.

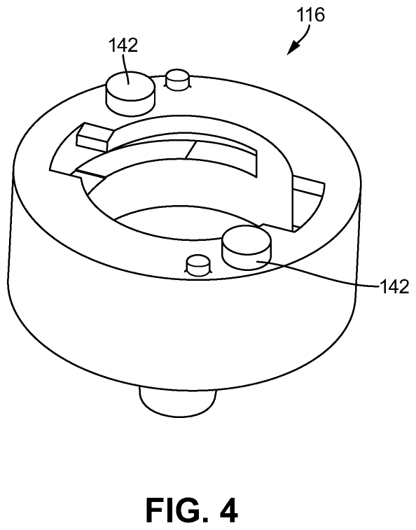

In some embodiments, the controller 112 can include a hall effect sensor 144 that is positioned adjacent the controller coupling interface 115. As shown in FIG. 4, some embodiments of the band coupling interface 116 can include one or more magnets 142 that can be sensed by the hall effect sensor 144, which is in communication with the processor 134. As such, the hall effect sensor 144 can detect the one or more magnets 142 when the band coupling interface 116 is coupled to the controller coupling interface 115 to form the coupling interface 114. Such detection by the hall effect sensor 144 can be communicated to the processor 134 thereby initiating, for example, pressure sensing by the pressure sensor 132.

The wearable compression system 100 can include a variety of coupling interfaces 114 for releasably coupling the controller 112 to the inflatable band 110. Various embodiments of the coupling interfaces 114 are described herein.

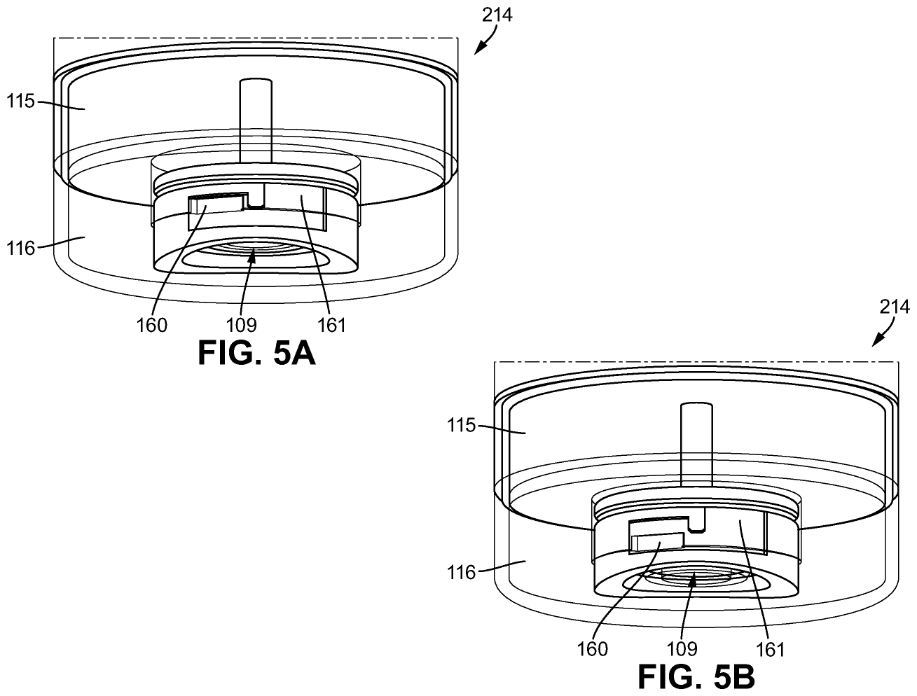

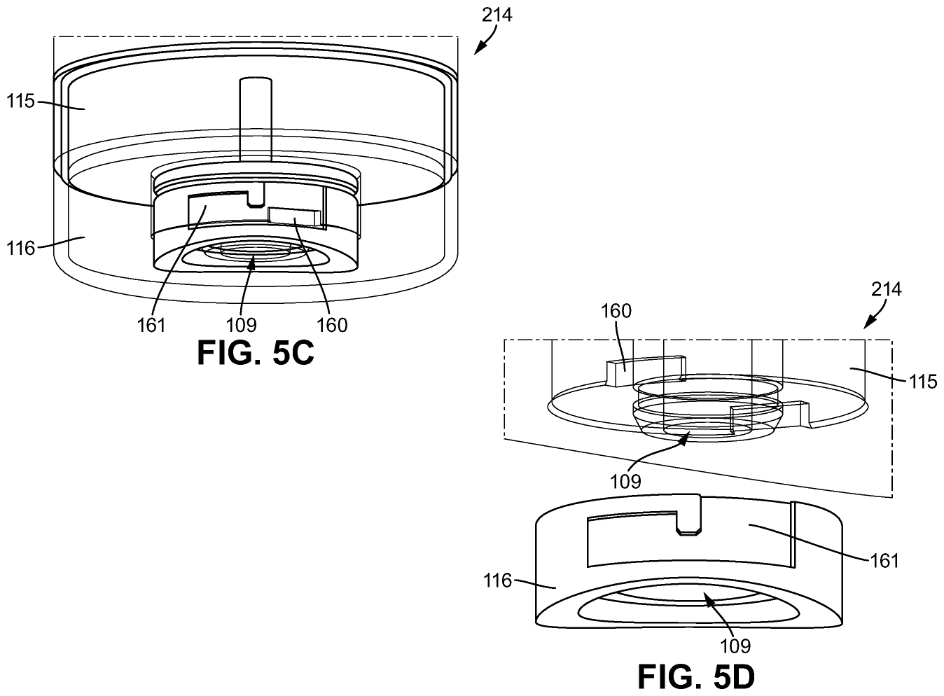

FIGS. 5A-5D illustrate an embodiment of a coupling interface 214, including an embodiment of a controller coupling interface 115 and an embodiment of a band coupling interface 116. For example, the controller coupling interface 115 may include a first coupling feature 160 including an extrusion or tab for interlocking with a second coupling feature 161 of the band coupling interface 116. As shown in FIG. 5D, the second coupling feature 161 can include a recess extending circumferentially along an outer wall of the band coupling interface 116 that can receive and secure the first coupling feature 160 therealong.

For example, FIG. 5A shows the first coupling feature 160 in a locked position, including the first coupling feature 160 within an indented slot of the second coupling feature 161. In the locked position, for example, an air-tight seal can be formed between the controller coupling interface 115 and the band coupling interface 116.

In some embodiments, the band coupling interface 116 and the controller coupling interface 115 can be unlocked and uncoupled relative to each other by guiding the first coupling feature 160 along the second coupling feature 161, such as shown in FIGS. 5A-5C. The first coupling feature 160 can slide along the second coupling feature 161 until the first coupling feature 160 can be disengaged from the second coupling feature 161, as shown in FIG. 5D, thereby uncoupling the band coupling interface 116 from the controller coupling interface 115.

In some embodiments, as shown in FIGS. 5A-5B, to unlock the band coupling interface 116 from the controller coupling interface 115, the controller 112 can be pushed towards the inflatable band 110, for example, to cause the first coupling feature 160 to move along and disengage from the second coupling feature 161. For example, a spring can be integrated in the coupling interface 214 to allow such transition when a force is applied to the controller 112 for uncoupling the controller 112 from the inflatable band 110.

As shown in FIG. 5C the controller 112 can be twisted to unlock the controller 112 from the inflatable band 110. For example, the first coupling feature 160 may travel along the recessed second coupling feature 161 when the controller 112 is pushed towards the inflatable band 110 and twisted. Other mechanisms for securely coupling and uncoupling the controller 112 from the inflatable band 110 is within the scope of this disclosure.

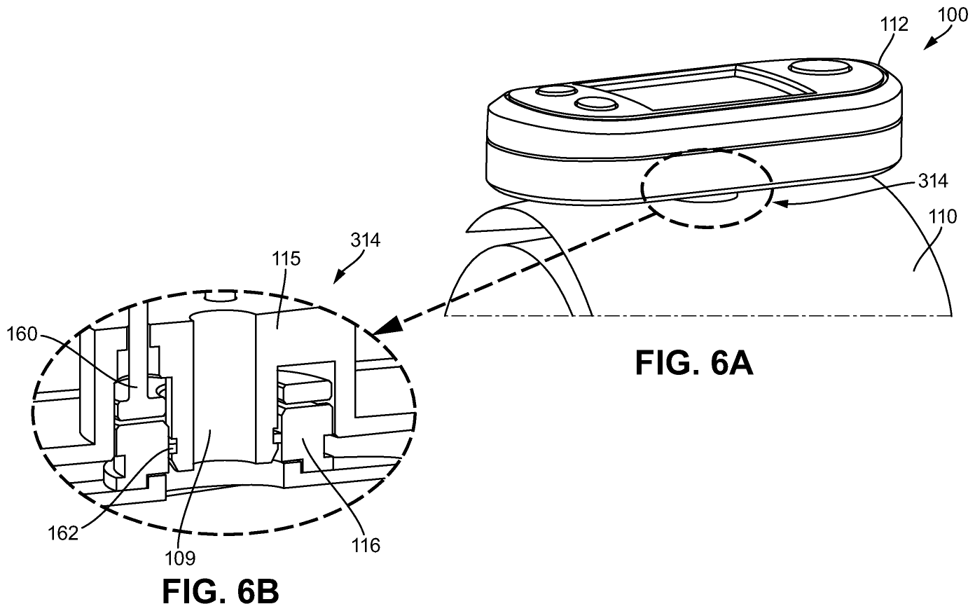

FIGS. 6A-6B illustrate an embodiment of a wearable compression system 100 including an embodiment of a coupling interface 314 including an embodiment of the controller coupling interface 115 and an embodiment of the band coupling interface 116 that can be releasably coupled together, as discussed above. Additionally, the controller coupling interface 115 can include an embodiment of the first coupling feature 160 including a spring plate that can releasably couple to and/or engage with a second coupling feature 161. As shown in FIG. 6B, the second coupling feature 161 can include an inner passageway including a fluid seal or o-ring 162 that can receive a part of the controller coupling feature 115 for forming a sealed airflow pathway 109 (e.g., extending between the pump 130 of the controller 112 and bladder 120 of the inflatable band 110). For example, the spring plate can ensure the controller 112 stays locked to the inflatable band 110 by exerting a force that keeps the first coupling feature 160 coupled to the second coupling feature 161, as shown in FIGS. 5A and 5C.

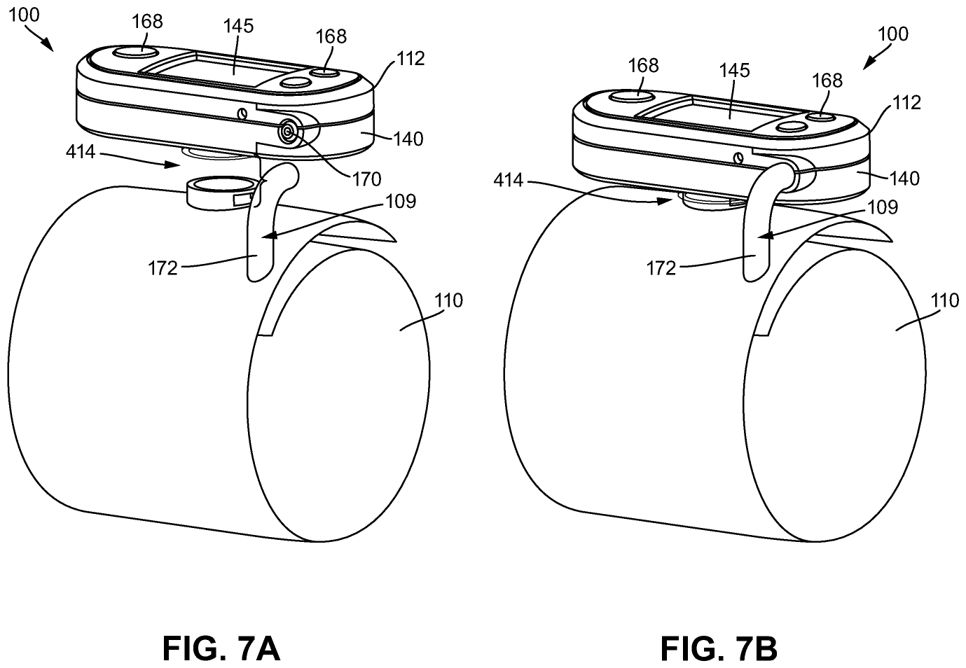

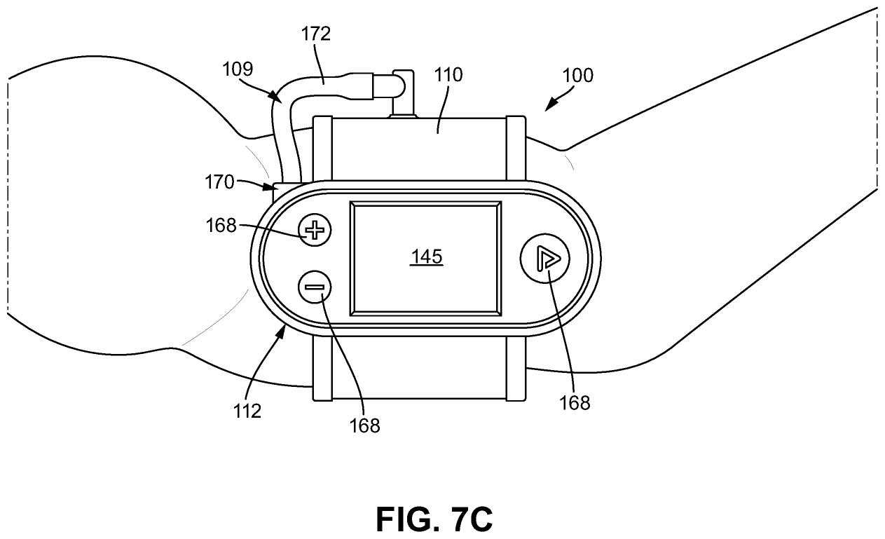

FIGS. 7A-7C illustrate another embodiment of a wearable compression system 100 including an external tube 172 that extends between an outlet port 170 of the controller 112 and the inflatable band 110. For example, the external tube 172 can provide the airflow pathway 109 between the pump 130 of the controller 112 and the bladder 120 of the inflatable band 110. As such, the coupling interface 414 shown in FIGS. 7A and 7B can be void of the airflow pathway 109.

In some embodiments, the external tube 172 can include a one-way valve. The one-way valve may be configured to be turned on and off by the controller 112. The external tube 172 may include a restrictor to limit the airflow through the external tube 172. The external tube 172 may be made of a flexible material to allow movement between the controller and the inflatable band.

The outlet port 170 along the housing 140 of the controller 112 can allow fluid (e.g., air) to pass between the housing and the external tube 172. The outlet port 170 can be positioned along any side of the housing 140 without departing from the scope of this disclosure. In some embodiments, the external tube 172 and/or the outlet port 170 can include a reliable coupling feature, such as a threaded features, for allowing the external tube 172 to releasably couple to the controller 112, such as to allow the controller 112 to uncouple from the inflatable band 110.

In some embodiments, the external tube 172 is configured to releasably connect to the inflatable band 110. For example, the inflatable band 110 may have an inflatable band opening at an exterior surface through which gaseous or liquid matter passes. The inflatable band opening may enable a gaseous or liquid matter to pass between the external tube 172 and the inflatable band 110. For example, the tube opening may include a threaded ring to couple to the external tube 172. Additionally, and/or alternatively, the inflatable band opening may include holes through which a tab and spring (of the external tube 172) may fit. Alternatively, and/or additionally, the inflatable band opening may be configured to latch to the tube opening via an insert, a screw, a magnet, a port, and/or the like.

As shown in FIG. 3A, the controller 112 can include a user interface 145, such as for observing and programming settings of the wearable compression system 100, as will be discussed in greater detail below. For example, the controller 112 can monitor and regulate pressure inside of the inflatable band 110. The controller 112 may provide a user interface 145, such as shown in FIG. 9A, to display monitored readings and enable the user to select operation configurations.

For example, as discussed above, the controller 112 can regulate the pressure inside of the inflatable band 110. The controller 110 may apply programmed logic (e.g., using processor 134) to determine whether to adjust the pressure inside of the inflatable band 110. This programmed logic may include upper pressure thresholds, lower pressure thresholds, and desired operating thresholds. For example, the controller 112 may determine that under no circumstances should the pressure inside of the inflatable band 110 exceed a compression resulting in 100% of limb occlusion pressure. In another example, a desired operating threshold may be 30% of limb occlusion pressure. The controller 112 may regulate the pressure inside of the inflatable band 110 by adjusting the amount of fluid (e.g., air) inside the interior volume of the inflatable band 110. For example, the controller 112 may instruct the pump 130 to add additional air to the inflatable band 110 when the measured pressure is lower than a threshold. The controller 112 may regulate the pressure inside of the inflatable band 110 based on readings other than pressure. For example, the controller 112 may regulate the pressure inside of the inflatable band 110 based on an acceleration, a duration of the exercise, a blood pressure, a surface area covered by the inflatable band, the amount of resistance applied to the limb or extremity, and/or a diameter of the limb around which the inflatable band 110 is wrapped.

For example, the pump 130 can be a DC motor pump, a piezo pump, and/or the like. As shown in FIGS. 3C and 3D, the controller 112 can include a pressure relief valve 138 that can release pressure from the inflatable band 110. For example, the controller 112 may determine the number of reps a user preforms using the pressure sensor 132 and/or an accelerometer. The pressure relief valve 138 may release pressure when the user contracts their muscle to actively regulate the pressure, such as maintain the pressure within a desired pressure range that safely and effectively restricts blood flow along the extremity. For example, the pressure relief valve can be instructed by the processor 134 to release pressure from the bladder 120 as a result of a pressure reading by the pressure sensor 132 that exceeds a programmed pressure threshold. Alternatively, and/or additionally, the pressure relief valve 138 may act as an emergency safety valve that may be activated by a user. For example, this threshold value may include 200 mmHg or any other pressure levels recommended by a medical practitioner. In some implementations, the pressure relief valve 138 may deposit air into a controller reservoir within the housing 140. The controller reservoir may be used to regulate the pressure in the inflatable band 110, such as when the user relaxes or contracts an associated muscle. The pressure relief valve 138 may be a solenoid valve. For example, the reservoir can hold expelled air and return some of the expelled air when the pressure drops. This can reduce the pressure peaks, increase the pressure valleys, and thereby reduce fluctuation in pressure.

The pressure sensor 132 and the relief valve 138 can each be in fluid communication with the airflow pathway 109 that extends between the pump 130 and the inflatable band 110, such as the bladder 120. For example, the airflow pathway 109 can extend between the pump 130 and the inflatable band 110 via an embodiment of the coupling interface 114 or via the outlet port 170 and external tubing 172. The processor 134 can communicate with one or more valves 139 along the airflow pathway 109 that assist with controlling fluid flow along the airflow pathway. For example one or more valves 139 can be positioned adjacent the pressure sensor 132, the relief valve 138, and the pump 130 and can each be independently instructed to open (e.g., allow fluid flow therethrough) or close (e.g., prevent fluid flow therethrough). For example, during inflation of the inflatable band 110 in order to increase the pressure in the inflatable band 110, the valve adjacent the relief valve 138 can be closed. Additionally, the valve adjacent the pressure sensor can be open in order to allow the pressure sensor to monitor the pressure within the inflatable band 110, such as within the bladder 120 (e.g., by measuring the pressure along the airflow pathway 109).

The processor 134 may be powered by the battery 126. The battery 136 may be a rechargeable battery configured to be recharged by a solar power source or a kinetic power source. The battery 136 may also power the pump 130, the relief valve 138, and the pressure sensor 132. In some implementations, the controller 112 may include a Bluetooth connection module, a Wi-Fi module, or another near-field communication method.

As shown in FIGS. 3A and 7C, the controller 112 may include a user interface 145 through which monitored readings and settings for controlling the inflatable band 110 are presented. The user interface 145 may include a screen to display multiple readings relating to the inflatable band 110. For example, the user interface 145 may display the duration of the workout and the current occlusion pressure. As discussed above, the user interface 145 of the controller 112 can be configured to rotate the display, such as based on an orientation of the controller relative to either the user's arm and the ground. For example, the controller can include an IMU sensor that can rotate the display to allow the user to view the display when in a variety of orientations and positions.

FIGS. 9A and 9B show the user interface 145 including a touchscreen. The touchscreen feature of the user interface 145 can enable the user to select settings related to the inflatable band 110. For example, the user interface 145 may present a menu from which the user may select a percentage of occlusion pressure, as shown in FIG. 9B, thereby enabling the user to set pressure thresholds of the interior volume of the inflatable band 110 (e.g., the bladder 120). Alternatively, and/or additionally, as shown in FIG. 7C, one or more tactile buttons 168 may enable the user to select settings related to the inflatable band 110. For example, the tactile buttons 168 may enable the user to select an occlusion pressure or to open a valve to release pressure from the inflatable band 110. Any number of selectable features and settings associated with the wearable compression system 100, including any features and settings discussed herein, can be modified and controlled via the touchscreen user interface 145 and/or tactile buttons 168.

In some implementations, the controller 112 may send instructions and feedback to the user via the user interface 145. For example, the instructions and feedback can include information to aid the user to complete an exercise safely and effectively. For example, the controller 112 may present a warning to the user when the controller 112 detects pressure levels in the inflatable band 110 that are too high or too low. For example, the controller 112 may also provide a warning to the user when the controller 112 detects the user is performing the exercise too quickly, such as a sensed acceleration by an accelerometer associated with the processor 134. For example, the wearable compression system 100 can warn or alert a user regarding one or more conditions via the user interface 145, an audible noise provided by the controller 112, tactile feedback (e.g., vibration), and/or a light that can be illuminated (in one or more colors).

As shown in FIG. 3B, a light strip 150 can extend circumferentially along a side of the housing 140 of the controller 112, which can illuminate to alert the user of one or more conditions (e.g., pressure in the inflatable band 110 is too low/high, an exercise is being performed incorrectly, etc.). For example, such circumferential lighting provided by the light strip 150 along the side of the housing 140 can allow the user to sufficiently notice and see the illuminated light strip 150, such as when the wearable compression system 100 is coupled to an extremity, including during exercise. The light strip 150 can include one or more LED lights and be configured to illuminate in a variety of different ways (e.g., blink, dim, etc.) and/or colors to provide various alerts to the user.

In some embodiments, the controller 112 can provide one or more images to the user interface 145 providing directions on how to perform an exercise using proper technique and form. The controller 112 may store information related to how the workout was performed and whether any warnings were presented to the user, such as the user was alerted to performing an exercise too rapidly or the pressure exceeded a programmed threshold. In some implementations, a personal trainer may provide or select a protocol that the controller 112 can present to the user to instruct the user how the exercise is properly performed.

FIG. 7C shows the wearable compression system 100 coupled to a human arm, in accordance with some example embodiments. For example, the wearable compression system 100 can apply pressure to a bicep of an arm thereby restricting blood flow at least along the bicep. For example, such restricting of the blood flow along the bicep can be performed at least while the user is performing an exercise with the arm having the wearable compression system 100 attached. Such restriction in blood flow can allow the user to perform lighter exercises while achieving at least approximately a same amount of strength and/or muscle gain. The lighter exercises (e.g., less resistance, less weight) can prevent over-use of one or more joints, as well as reduce injuries compared to a greater strength training for conventionally achieving greater strength and muscle gain.

The wearable compression system 100 can be controlled (e.g., increase/decrease applied pressure, duration, etc.) via one or more of a variety of ways, including various devices. For example, a mobile application can be utilized on a mobile device that allows a user to control the wearable compression system 100. In some embodiments, the wearable compression system 100 can be controlled remotely, such as using a mobile device including the mobile application for controlling the wearable compression system 100.

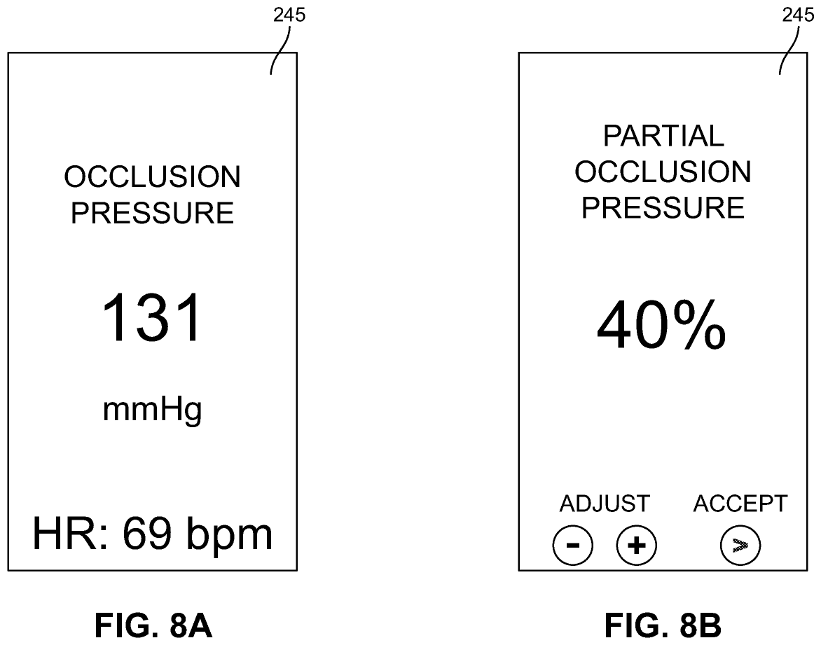

FIG. 8A-8B illustrate an exemplary user interface 245 of a mobile application for communicating with the controller 112 of a wearable compression system 100, in accordance with some example embodiments. A mobile device may include a Bluetooth connection module, a Wi-Fi module, or another near-field communication method to communicate with the controller 112.

The mobile user interface 245 may display monitored readings and settings for controlling the inflatable band 110. The mobile user interface 245 may include a mobile touchscreen to display readings relating to the inflatable band. For example, the mobile touchscreen may display the duration of the workout and the current occlusion pressure. The mobile touchscreen may enable the user to select settings related to the inflatable band. For example, the touchscreen may present a menu from which the user may select a percentage of occlusion pressure, enabling the user to set pressure thresholds of the interior volume. The touchscreen may enable the user to manually control the pressure of the inflatable band. The touchscreen may enable the user to select an occlusion pressure or to open a valve to release pressure from the inflatable band.

The mobile user interface may be standalone or may run in conjunction with a mobile application to control the controller 112. The mobile application may display results and statistics of a workout and enable a third party (e.g., physical therapist or personal trainer) to gain access to the results and statistics. The mobile application may instruct the user to procure the limb occlusion pressure. For example, the mobile application may direct the user to determine limb occlusion pressure by placing the inflatable band and any extra sensors where needed on the desired limb, laying down (if deemed necessary), and pressing a limb occlusion pressure detection button on the mobile application. The mobile application should be able to store the last determined limb occlusion pressure for a user and be able to accommodate multiple users. The mobile application may provide medical warnings to users. For example, the mobile application may display warnings to high blood pressure individuals and recommend speaking with a physician before using the wearable compression system 100.

The mobile user interface 245 may include preset protocols and the ability to create new protocols. Preset protocols may be accessed from the internet through the mobile application. The mobile user interface 245 may display how the exercise is performed in the form of a picture, a gif, or a video. The mobile user interface 245 may enable the user to input weight, reps, sets, and a level of difficulty. The mobile user interface 245 may enable the user to create and save a user profile. A user profile may include a user weight, height, age, medical conditions, past performance records, statistics, and/or the like. The mobile user interface 245 may display the set pressure, live pressure in the band, and timer for exercise purposes. The mobile user interface 245 may enable an emergency stop to be selected at any time to deactivate the wearable compression system 100 or reduce pressure in the inflatable band 110.

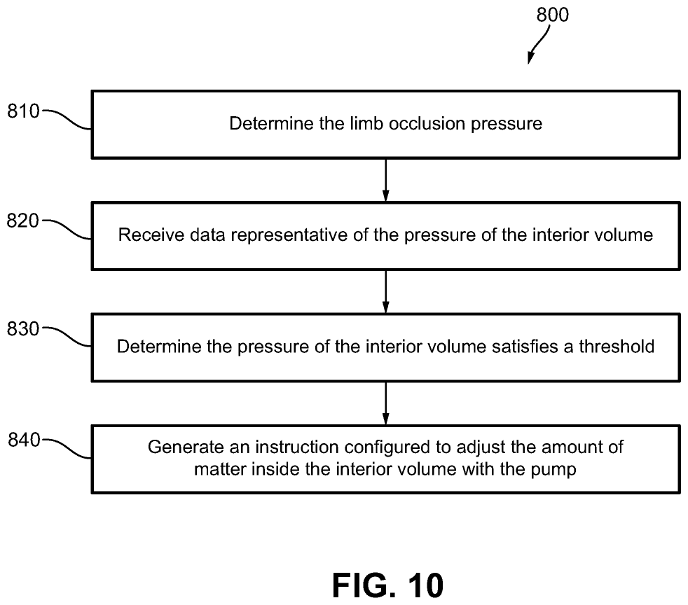

FIG. 10 shows a flowchart illustrating a process for controlling a pressure inside of an embodiment of the inflatable band 110, such as controlling a pressure within the bladder 120 for applying a pressure to user for restricting blood flow. The pressure regulation flowchart process 800 may be supervised and/or performed by the wearable compression system 100.

At 810, an embodiment of the processor 134 determines an extremity or limb occlusion pressure. For example, the limb occlusion pressure may be determined by doppler ultrasound downstream of the inflatable band 110, photoplethysmogram downstream of the inflatable band 110, the amplitude of the pulse detected by a pressure sensor, Auscultatory method (Korotkoff sounds), oscillometry method, and/or the geometry of the limb. Using photoplethysmography, a band (bracelet/anklet) can be put on distal end away from the inflatable band 110 and can monitor for the pulse waveform. Once that waveform disappears, the limb occlusion pressure can be determined.

At 820, the processor 134 may receive from the pressure sensor 132, data representative of the pressure of the interior volume of the inflatable band 110, such as within the bladder 120. For example, the processor 134 may receive data that the pressure of the interior volume is 10% of the limb occlusion pressure.

At 830, the processor 134 may determine, based on the data representative of the pressure of the bladder 120, the pressure of the bladder 120 satisfies a threshold. For example, the processor 134 may determine the 10% limb occlusion pressure is below the 20% threshold of the limb occlusion pressure.

At 840, the processor 134 may generate, in response to the pressure of the bladder 120 not satisfying the threshold (e.g., the pressure is too low), an instruction that is sent to the pump 130. The instruction can instruct the pump 130 to adjust the amount of fluid (e.g., air) inside the bladder 120. For example, the processor 134 may activate the pump 130 to increase the amount of air inside the interior volume of the bladder 120. The processor 134 may instruct the pump 130 to turn off when the desired pressure is sensed, such as by an embodiment of the pressure sensor 132.

As discussed above, the wearable compression system 100 can be controlled and/or programmed by a separate device (e.g., a mobile device). For example, a mobile device can directly or wirelessly program the wearable compression system 100 to run various protocols, such as protocols related to exercise and/or therapy movements performed while wearing and using the wearable compression system 100. For example, the wearable compression system 100 can be programmed to detect and/or track various movements performed by the user to determine a performance by the user associated with a selected protocol. The wearable compression system 100 can thus assist a user with performing one or more exercise and/or physical therapy movements, as well as track and/or sense one or more associated characteristics (e.g., reps performed, occlusion pressure, etc.). Such tracked or sensed information can be saved either on the mobile device or the wearable compression system 100, such as for future reference by the user, exercise trainer, and/or healthcare professional.

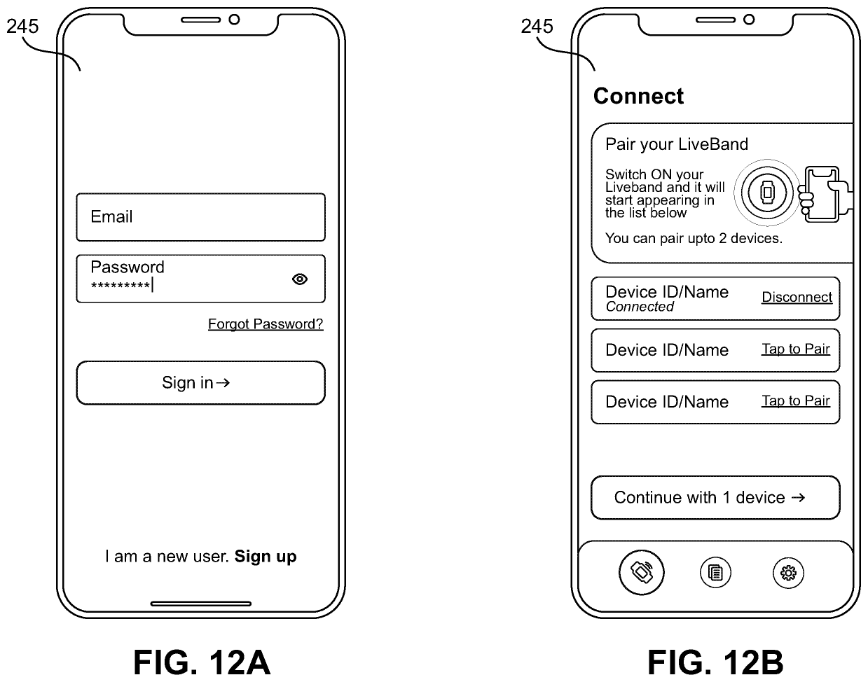

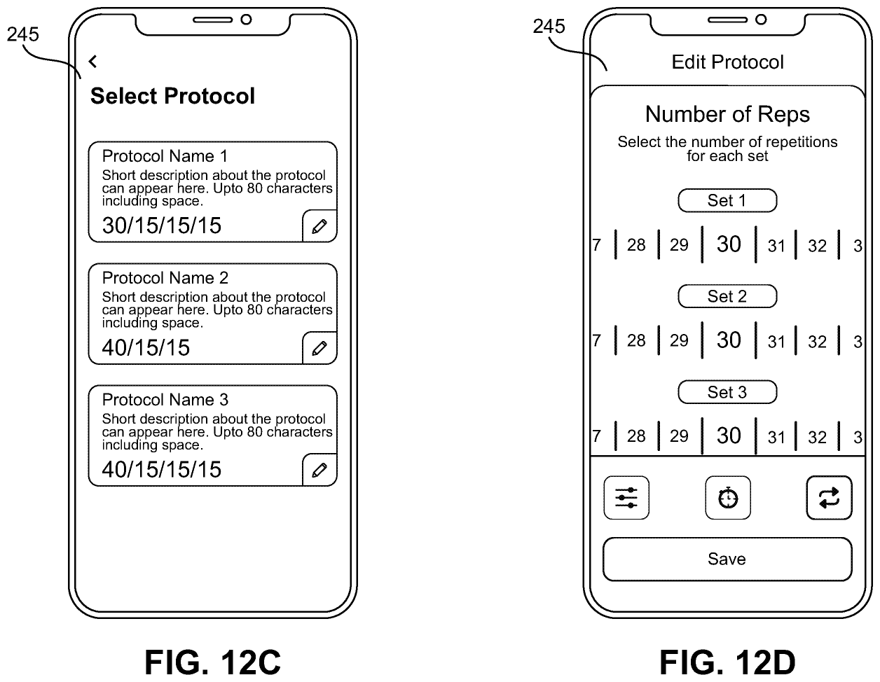

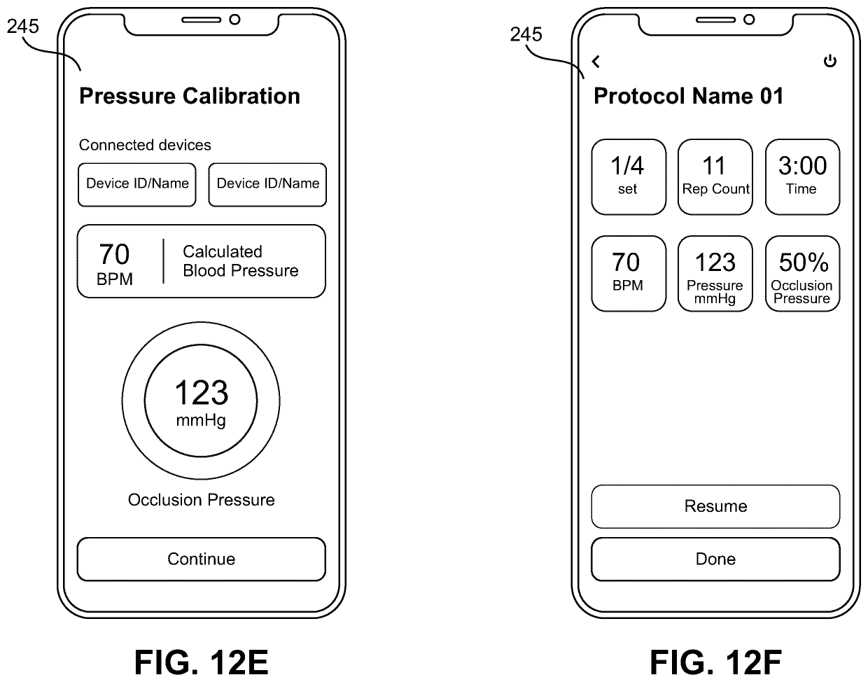

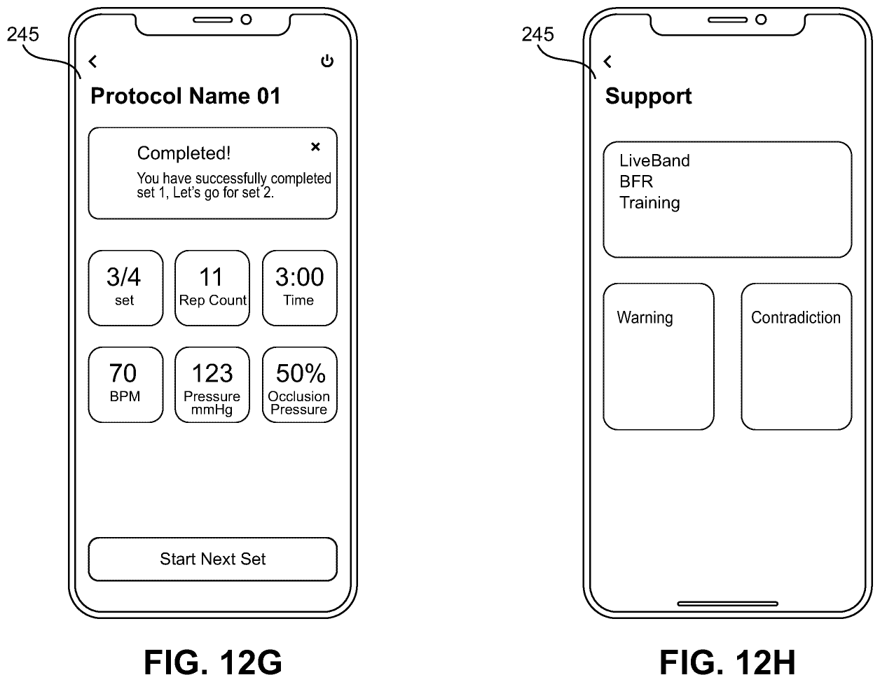

FIGS. 12A-12H illustrate an example user interface and programming of the wearable compression system 100, including programming and running a protocol via a mobile device. The protocol can define at least one exercise or physical therapy movement to be performed by an extremity of a user of the wearable compression system 100, such as for achieving increased strength of the extremity as a result of at least reducing blood flow along the extremity. For example, FIGS. 12A-12H provide example displays provided by the user interface for at least programming user information, including information related to one or more protocols. Although the following describes an example of programming the wearable compression system 100 using a mobile device, one or more of the following steps can be performed directly at the wearable compression system 100, such as via a user interface of the controller 112.

As shown in FIG. 12A, the mobile user interface 245 can include a touchscreen display including a user login display. The user login display can allow a user to access and program user information to the wearable compression system 100, such as user characteristics (e.g., limb occlusion pressure) and protocols tailored for the user (e.g., exercise and/or physical therapy routines to be performed while wearing the wearable compression system 100).

As shown in FIG. 12B, the mobile user interface 245 can display selectable icons and information for pairing the mobile device with an embodiment of the wearable compression system 100. For example, the mobile device can wirelessly pair with the controller 112 of the wearable compression system 100 to program one or more protocols and/or view user data, such as past performance characteristics, limb occlusion pressure, etc.

As shown in FIG. 12C, the mobile user interface 245 can display one or more editable protocols. For example, a protocol selected for editing can define one or more aspects of an exercise or physical therapy movement or series of movements to be performed by a user. The editable protocol can also define one or more characteristics associated with the user, such as limb occlusion pressure and movements to be performed during use of the wearable compression system 100. One or more protocols can be programmed and saved, such as through the mobile device, which can thereafter be selected by a user for controlling and sensing the wearable compression system 100 in accordance with the programmed parameters. For example, a first protocol can be selected by a user that can control an amount of occlusion pressure applied over a programmed amount of time and/or during a programmed number of repetitions of a movement. The protocol can also be configured to calibrate an appropriate occlusion pressure or limb occlusion pressure to be applied to the user, such as by sensing a blood pressure of an extremity of the user, including sensing a blood pressure after pressure applied to the extremity.

As shown in FIG. 12D, the mobile user interface 245 can display an editable number of movement repetitions that are to be performed by a user of the wearable compression system during each set, which can be defined in a programmed protocol selected by the user. One or more sensors of the wearable compression system 100 (including any described herein) can be used to detect and track the number of repetitions of a movement performed by the user. For example, the selected protocol can detect and track movement by the user in accordance with the selected protocol. Additionally, the wearable compression system 100 can provide one or more status updates to the user in response to such tracked data, such as illuminate an indicator (e.g., illuminate light strip 150), to alert the user as to a completion of a set of repetitions in accordance with the protocol. Other status updates and programmed characteristics associated with a protocol can be defined and performed by the wearable compression system 100 without departing from the scope of this disclosure.

As shown in FIG. 12E, the mobile user interface 245 can display a calibrating occlusion pressure of a user of the wearable compression system, which can assist with defining an appropriate occlusion pressure for achieving one or more of the benefits associated with the wearable compression system 100 described herein, such as improved strength of an extremity of a user. In some embodiments, one or more additional features and sensors can be in communication with the mobile device for sensing characteristics of the user, such as blood pressure at a location away from the wearable compression system 100. For example, the pressure calibration display can be used to initiate at least one blood pressure sensor and applying various amounts of occlusion pressure along an extremity of a user. For example, a processor of the wearable compression system 100 can analyze the sensed blood pressure and occlusion pressure to determine an appropriate limb occlusion pressure, as well as determine a percentage of occlusion pressure that is appropriate for the user.

As shown in FIG. 12F, the mobile user interface 245 can display a summary of programmed parameters or characteristics associated with a programmed protocol of the wearable compression system 100. For example, the programmed parameters or characteristics can include one or more of a number of repetitions and sets of a movement to be performed by a user, a time to complete such movements (or other time based characteristics), a blood pressure, a heart rate, and an occlusion pressure (e.g., a percentage of a limb occlusion pressure). Any one or more of the programmed parameters can be achieved by sensing characteristics associated with the user (e.g., blood pressure) and actively adjusting one or more parameters (e.g., applied pressure to the associated extremity of the user) in order to achieve the programmed protocol parameters.

As shown in FIG. 12G, the mobile user interface 245 can display a status of programmed protocol, such as an indication of a completion of the protocol (e.g., successful completion or unsuccessful completion).

As shown in FIG. 12H, the mobile user interface 245 can display a system support display for the wearable compression system 100. For example, the system support display can provide a plurality of information associated with the user of the wearable compression system 100, such as addressing warnings/alerts provided by the system. Additionally, the system support display can provide access to support for training with the wearable compression system 100, such as exercise and/or physical therapy movements that can be performed by the user.

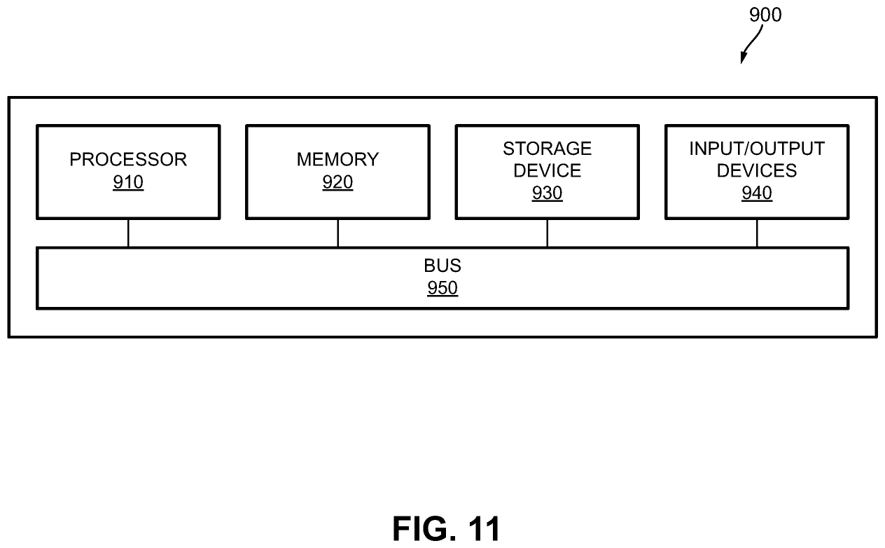

FIG. 11 depicts a block diagram of an example computing system 900 of a wearable compression system 100, in accordance with some embodiments. For example, the computing system 900 can be at least partially contained within the controller 112.

As shown in FIG. 11, the computing system 900 can include a processor 910 (such as processor 132 of FIG. 3C), a memory 920, a storage device 930, and input/output devices 940. The processor 910, the memory 920, the storage device 930, and the input/output devices 940 can be interconnected via a system bus 950. The processor 910 is capable of processing instructions for execution within the computing system 900. Such executed instructions can implement one or more components of, for example, the wearable compression system 100. In some embodiments, the processor 910 can be a single-threaded processor. Alternatively, or additionally, the processor 910 may be a multi-threaded processor. The processor 910 is capable of processing instructions stored in the memory 920 and/or on the storage device 930 to display graphical information for a user interface provided via the input/output device 940.

The memory 920 is a computer-readable medium such as volatile or non-volatile that stores information within the computing system 900. The memory 920 can store data structures representing configuration object databases, for example. The storage device 930 is capable of providing persistent storage for the computing system 900. The storage device 930 can be a data cloud storage, a hard disk device, an optical disk device, a solid-state drive, and/or other suitable persistent storage means. The input/output device 940 provides input/output operations for the computing system 900. In some example embodiments, the input/output device 940 includes a keyboard and/or pointing device. In various implementations, the input/output device 940 includes a display unit (e.g., user interface 145, 245) for displaying graphical user interfaces.

According to some example embodiments, the input/output device 940 can provide input/output operations for a network device. For example, the input/output device 940 can include Ethernet ports or other networking ports to communicate with one or more wired and/or wireless networks (e.g., a local area network (LAN), a wide area network (WAN), the internet).

In some example embodiments, the computing system 900 can be used to execute various interactive computer software applications that can be used for organization, analysis and/or storage of data in various formats. Alternatively, the computing system 900 can be used to execute any type of software application. These applications can be used to perform various functionalities, e.g., applied pressure regulation (e.g., generating, controlling, modifying, etc.), computing functionalities, communications functionalities, etc. The applications can include various add-in functionalities or can be standalone computing items and/or functionalities. Upon activation within the applications, the functionalities can be used to generate the user interface provided via the input/output device 940. The user interface can be generated and presented to a user by the computing system 900 (e.g., on user interface 145, 245).

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program item, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) or organic light emitting diode (OLED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input devices include touch screens or other touch-sensitive devices such as single or multi-point resistive or capacitive track pads, voice recognition hardware and software, optical scanners, optical pointers, digital image capture devices and associated interpretation software, and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

The subject matter described herein can be embodied in systems, apparatus, methods, and/or articles depending on the desired configuration. The implementations set forth in the foregoing description do not represent all implementations consistent with the subject matter described herein. Instead, they are merely some examples consistent with aspects related to the described subject matter. Although a few variations have been described in detail above, other modifications or additions are possible. In particular, further features and/or variations can be provided in addition to those set forth herein. For example, the implementations described above can be directed to various combinations and sub combinations of the disclosed features and/or combinations and sub combinations of several further features disclosed above. In addition, the logic flows depicted in the accompanying figures and/or described herein do not necessarily require the particular order shown, or sequential order, to achieve desirable results. Other implementations may be within the scope of the following claims.

1. A wearable compression control system for controlling an applied pressure against an extremity to restrict blood flow along the extremity of a user performing an exercise or undergoing a therapy, the wearable compression control system comprising:
an inflatable band including a coupling feature for securing the inflatable band in a position along the extremity such that the inflatable band extends circumferentially around the extremity, the inflatable band including a bladder extending along a length of the inflatable band, the bladder including an expandable inner volume for receiving fluid to cause the inflatable band to increase in thickness and increase the applied pressure against the extremity; and
a controller that releasably couples to the inflatable band, the controller comprising:
a processor including a programmed pressure range, the processor configured to actively maintain the applied pressure within the programmed pressure range throughout the user performing the exercise or the user undergoing the therapy;
a pressure sensor positioned along an airflow pathway that is in fluid communication with the bladder, the pressure sensor sensing a pressure within the bladder that indicates an amount of the applied pressure against the extremity for at least restricting blood flow, the processor configured to receive sensed pressure data from the pressure sensor during performance of the exercise or therapy;
a pump in fluid communication with the airflow pathway, the pump configured to continuously receive instructions from the processor to provide fluid to the bladder for increasing the pressure within the bladder to reach the programmed pressure range throughout the user performing the exercise or the user undergoing the therapy; and
a relief valve configured to receive instructions from the processor to release fluid from the bladder to thereby reduce the pressure within the bladder when the sensed pressure is above the programmed pressure range.

2. The wearable compression system of claim 1, wherein the controller includes a controller coupling interface and the inflatable band includes a band coupling interface, the controller coupling interface including a first coupling feature that releasably engages with a second coupling feature of the band coupling interface for releasably coupling the controller to the inflatable band.

3. The wearable compression system of claim 2, wherein the coupling of the band coupling interface to the controller coupling interface forms a part of the airflow pathway extending between the pump and the bladder.

4. The wearable compression system of claim 2, wherein the band coupling interface includes a magnet and the controller includes a hall effect sensor for detecting when the band coupling interface is adjacent the controller coupling interface.

5. The wearable compression system of claim 1, wherein the programmed pressure range is approximately 40% to approximately 80% of a limb occlusion pressure associated with the user.

6. The wearable compression system of claim 1, wherein the controller further comprises a user interface.

7. The wearable compression system of claim 6, wherein the user interface includes a touchscreen.

8. (canceled)

9. The wearable compression system of claim 1, wherein the controller further includes an accelerometer that measures acceleration of the wearable compression system during the user performing the exercise or the user undergoing the therapy, the processor configured to analyze the measured acceleration for determining whether the exercise was performed in accordance with one or more parameters.

10. The wearable compression system of claim 1, wherein the controller further includes a light strip extending along at least one side of a housing of the controller, the light strip providing an indication of one or more conditions of the wearable compression system and/or the user.

11. A method of a wearable compression system, the method comprising:
sensing, by a pressure sensor positioned along a fluid pathway of the wearable compression system, a first sensed pressure indicating a first pressure within an inflatable band of the wearable compression system, the inflatable band extending circumferentially around an extremity of a user;
receiving, at a processor of a controller of the wearable compression system, the first sensed pressure;
comparing, at the processor, the first sensed pressure against a programmed pressure range, the programmed pressure range including a lowest pressure for achieving blood flow restriction by the inflatable band along a part of the extremity;
activating, when the processor determines the first sensed pressure is below the programmed pressure range, a pump of the wearable compression system to deliver fluid to the inflatable band for increasing the pressure within the inflatable band;
monitoring, by the processor and during activation of the pump, sensed pressure data from the pressure sensor;
deactivating, by the processor, the pump when the processor receives a second sensed pressure that is greater than the programmed pressure range;
monitoring, by the processor and during performance of an exercise using the extremity, sensed pressure data from the pressure sensor;
activating, when the processor determines a third sensed pressure is above the programmed pressure range, a relief valve to release fluid from the inflatable band; and
actively controlling, by the processor during performance of the exercise, the pressure within the inflatable band to maintain the pressure within the programmed pressure range.

12. The method of claim 11, wherein the controller is configured to be releasably coupled to the inflatable band.

13. The method of claim 11, wherein the actively controlling the pressure within the inflatable band during performance of the exercise allows the inflatable band to maintain blood flow occlusion during performance of the exercise.

14. The method of claim 11, wherein the programmed pressure range is approximately 40% to approximately 80% of a limb occlusion pressure associated with the user.

15. The method of claim 11, further comprising:
displaying, at a user interface of the wearable compression system, one or more characteristics associated with an amount of pressure applied to the extremity by the inflatable band.

16. The method of claim 15, wherein the user interface is a touchscreen.

17. The method of claim 11, wherein the controller includes a controller coupling interface and the inflatable band includes a band coupling interface, the controller coupling interface including a first coupling feature that releasably engages with a second coupling feature of the band coupling interface for releasably coupling the controller to the inflatable band.

18. The method of claim 17, wherein the coupling of the band coupling interface to the controller coupling interface forms a part of an airflow pathway extending between the pump and a bladder.

19. The method of claim 17, wherein the band coupling interface includes a magnet and the controller includes a hall effect sensor for detecting when the band coupling interface is adjacent the controller coupling interface.

20. The method of claim 11, wherein the controller further comprises an accelerometer that measures acceleration of the wearable compression system during performance of the exercise, the processor configured to analyze the measured acceleration for determining whether the exercise was performed in accordance with one or more parameters.

21. The method of claim 11, wherein the controller further comprises a light strip extending along at least one side of a housing of the controller, the light strip providing an indication of one or more conditions of the wearable compression system and/or the user.

22. The method of claim 11, further comprising calibrating a limb occlusion pressure of the user to determine the programmed pressure range.

23. The method of claim 11, further comprising
sensing a movement of the user and tracking a number of movements performed by the user.

24. The method of claim 23, further comprising maintaining the applied pressure within the programmed pressure during performance of the movements by the user.
